(19) United States
(12) Patent Application Publication
(10) Pub. No.: US 2022/0028291 A1
(43) Pub. Date: Jan. 27, 2022

(54) DEVICE FOR THE IMPLEMENTATION OF SERIOUS GAMES FOR THE PREVENTION AND/OR TREATMENT OF MENTAL DISORDERS

(71) Applicant: Sympatient GmbH, Hamburg (DE)

(72) Inventors: Benedikt Reinke, Hamburg (DE); Christian Angern, Hamburg (DE); Julian Angern, Hamburg (DE)

(21) Appl. No.: 17/297,303

(22) PCT Filed: Nov. 26, 2019
(86) PCT No.: PCT/EP2019/082524

§ 371 Date: May 26, 2021

(51) Int. Cl. G09B 5/06; A61B 5/0205; A61B 5/024; A61B 5/08; A61B 5/1455; A61B 5/113; A61B 5/0531; A61B 5/11; A61B 5/16; A61B 5/318; A61B 5/378; A61B 5/398; A61B 5/00; A61B 7/00; A61B 5/395; A61B 5/38; G16H 20/70; G16H 50/20

(52) U.S. Cl. CPC G09B 5/06; A61B 2562/0219; A61B 5/02405; A61B 5/0816; A61B 5/14551; A61B 5/113; A61B 5/0531; A61B 5/1107; A61B 5/1114; A61B 5/163; A61B 5/318; A61B 5/378; A61B 5/398; A61B 5/4803; A61B 7/003; A61B 5/395; A61B 5/38; A61B 5/6803; A61B 5/749; G16H 20/70; G16H 50/20; A61B 5/0205

(57) ABSTRACT

Provided is a device for the implementation of serious games, i.e. for the presentation of digital games, which do not serve the purpose of entertainment, but the mediation of therapeutic content in the form of images, films, colors, sounds, etc., but may well contain such elements, for the treatment of mental disorders, whereby an authentic and credible, but also entertaining learning experience is the focus of interest in order to achieve a therapeutic result.

### CROSS-REFERENCE TO RELATED APPLICATIONS

The present application is a national phase application of International Application PCT/EP2019/082524 published as WO 2020/114831 A1 on Jun. 11, 2020 claiming priority to German Patent Application No. DE 10 2018 130 718.7 filed on Dec. 3, 2018. The aforementioned applications are hereby incorporated by reference in its entirety.

### BACKGROUND OF THE INVENTION

The present invention relates to a device for performing serious games for the prevention and/or treatment of mental disorders, as described by independent claim 1.

A mental disorder is a condition characterized by pathological changes in experience and behavior. It may be accompanied by deviations in perception, thinking, feeling, or even self-image or self-perception. Mental disorders are typically associated with significant personal distress or stress and problems in several areas of life.

An essential component of these disorders is also often a reduced self-regulatory competence. In this case, the affected persons can influence their disorder only with difficulty or not at all, even through increased efforts, self-discipline or willpower. Consequences of the psychological symptomatology are usually problems in coping with everyday life or impaired social relationships, e.g. due to difficulties in fulfilling social roles as before the illness.

Mental disorders come in many forms and are among the most widespread illnesses: The World Health Organization estimates that depression alone affects around 300 million people worldwide.

Mental disorders are among the most common reasons for consultations with general practitioners and in general medical practices. In Europe, anxiety disorders are in first place, followed by sleep disorders, depression, somatoform disorders, substance dependencies, ADHD in younger people and dementia in older people.

Today, mental disorders can be treated by the use of psychoeducation, occupational therapy and other methods in addition to the conventional use of psychotropic drugs and psychotherapy. The interplay between the affected person and his environment can also be of importance, so that this can be included in a treatment.

A major problem here is the availability of appointments and locations to carry out appropriate treatments. For example, a large number of psychological disorders go untreated simply because of the lack of appointments available, or it takes months to receive initial treatment. This is a major problem, especially in the care of patients outside metropolitan areas.

One conceivable solution to this problem could be to use modern communication technology, which could potentially significantly increase treatment rates and facilitate access to effective treatments. However, there is a lack of appropriate technical equipment with which to implement treatment approaches preventively, concomitantly or for follow-up treatment.

### SUMMARY OF THE INVENTION

Therefore, the object of the present invention is to provide a device for use in the treatment of mental disorders that allows patients to access therapeutic approaches easily and without spending time, that can serve as an initial access to professional therapeutic content, and that can help by accompanying existing therapies.

This problem is solved by a device having the features of the independent patent claim, wherein useful embodiments are described by the features as contained in the subclaims.

In this context, a device is envisaged for the implementation of serious games, i.e. for the presentation of digital games which do not serve the purpose of entertainment, but rather the mediation of therapeutic content in the form of images, films, colors, sounds, etc., but which may well contain such elements, for the treatment of mental disorders, whereby an authentic and credible, but also entertaining learning experience is the focus of interest in order to achieve a therapeutic result.

In accordance with the invention, the device for carrying out serious games thereby comprises
At least a facility for the representation of experiential spaces,
Sensors for physiological measurement of body functions,
Facilities for collecting and storing environmental data,
a module for recording and storing subjective raw data,
a module for recording and storing therapeutic guideline values,
Facilities for evaluating sensor and environmental data and raw subjective data to calculate feedback coefficients,
Facilities for automated interpretation of feedback coefficients, taking into account therapeutic benchmarks and deriving a feedback decision,
A module to control the facilities to display experiential spaces in response to the feedback decision,
Facilities for external monitoring of sensor data as well as feedback decisions, which may include interfaces to third party vendors, external devices, etc.; and
Facilities for external manipulation of feedback decisions.

The device according to the invention as described solves the problem by advantageously providing immersive therapy to patients who previously would have either not received treatment or received it only after a long delay. It provides access to mental disorder treatment for all by offering the potential to provide the severely underserved population of mentally ill patients with a new standard of care at a fraction of the cost of conventional therapies, particularly, though not limited to, for disorders such as: Neurotic, Stress and Somatoform Disorders (Anxiety, Compulsions), Affective Disorders (Depression and the like), Behavioral Disorders with Physical Disorders and Factors (Sleep and Sexual Dysfunction), Mental and Behavioral Disorders due to Psychotropic Substances (Substance Abuse, Addiction); Schizophrenia, Schizotypal and Delusional Disorders.

In a preferred embodiment, the device according to the invention can be designed in such a way that the devices for calculating feedback coefficients calculate them repetitively at defined time intervals, wherein the defined time intervals can comprise regular as well as randomly selected, i.e. random time intervals.

The sensors of the device as further described above, which may correspond to common sensors of different manufacturers, are preferably those for measuring psychophysiological activities, selected, though not limited, from the group:
Cardiovascular activities, such as heart rate, heart rate variability, respiration/oxygenation of blood via electrocardiogram (ECG) or optical sensor;
Brain activities, such as electrical events of cortical regions of the brain, electroencephalography (EEG);
Muscle activity, such as muscle tension, respiratory activity, electromyography;
Electrodermal activity, such as sweat, skin conductance;
Eye activities, such as eye movements and gaze direction, eye tracking optically and with electrooculography (EOG);
Motion activities, such as head movements, upper body movements via motion sensors; and
Measurement of voice pattern and breath sounds.

Further preferably, the devices for displaying experience spaces comprise elements for providing audiovisual content and/or sensory impulses, and at least one generator for audiovisual content may also be provided in addition.

In an equally preferred embodiment, a media library for storing audiovisual as well as sensory media resources is also provided for playback via the devices for displaying experience spaces, whereby certain media contents are assigned to certain feedback coefficients, i.e., bpsw. a certain image, sound, film, noise to a certain stress level.

In a further preferred embodiment of the device according to the invention, the devices for evaluating the sensor and environmental data and the subjective raw data for calculating feedback coefficients and the device for automated interpretation of the feedback coefficients and derivation of a feedback decision are each designed as a central device, including databases for subjective raw data, body function data and/or therapeutic reference values, to which a plurality of devices have access, whereby feedback coefficients as well as feedback decision are calculated on the basis of aggregated data of all connected devices. In this way, the data base, which is an essential building block for effective treatment, can be significantly broadened than if these facilities were only locally available and served only one device.

The device according to the invention may also be characterized in that the means for evaluating the sensor and environmental data and the raw data and for calculating feedback coefficients, and the means for automatically interpreting the feedback coefficients and deriving a feedback decision are designed such that an approximation to predefined target parameters is made by a systematic, iterative variation of the feedback decision in response to successive feedback coefficients.

Feedback coefficients can be entered into the system by therapists based on empirical values or scientific results via corresponding interfaces.

In another preferred embodiment of the device, the identification of the respective sensor data as well as the subjective raw data to calculate the feedback coefficients is performed by a self-learning pattern recognition system that accesses the data from the central devices, wherein the pattern recognition can be performed by AI methods, clustering analyses, machine learning, and/or the use of artificial neural networks in the sensors to identify patterns in the collected data such as heartbeats, respiration, brain activity, etc. The pattern recognition thereby checks and decides how to optimize a target parameter over the collected data from many devices connected to the central facilities. The device thereby varies the parameters over any number of persons using the connected devices and determines what the reactions of the persons are in order to determine the most efficient way to reach a goal (e.g. the stress level).

Pattern recognition can also be performed based on classification of digital images, videos, audio, and speech signals captured by the device based on, for example, low-level features such as edges or pixel attributes of captured images of the person being treated.

In a particularly preferred embodiment of the device, the means for displaying experiential spaces are designed as virtual reality (VR) goggles, augmented reality goggles, or mixed reality goggles, which are provided with corresponding sensors for physiological measurement of bodily functions. In this way, the device can be sent to patients to perform treatments.

The other elements and modules of the device can be combined in a mobile unit that can be connected to the goggles, so that the entire device can be used in a highly mobile manner.

Alternatively, the facilities for displaying experience spaces can be designed as simulators, i.e. stationary facilities equipped with the appropriate sensors for physiological measurement of bodily functions.

The device for inputting subjective raw data is preferably selected from the group for recording voice commands and/or ambient sounds, for recognizing gaze directions or for gesture recognition, for querying multiple choice questions, for querying binary statements, for specifying a range, for answering therapist questions, or the like.

The devices for analyzing the sensor and environmental data and the subjective raw data preferably comprise at least one device for analyzing speech and breathing sounds. In addition, the devices for the acquisition of environmental data may comprise light sensors, cameras, motion sensors, acceleration sensors as well as gyroscopes.

Finally, the device according to the invention may also be characterized by being locally or remotely connected to a plurality of devices of the same type via appropriate interfaces, for the presentation of an experience space common to all devices.

In particular, in a preferred embodiment, a software module may be provided for live transmission of the image from the experience room to a practitioner (and/or vice versa) and integration of voice-over-ip or the like for communication with a practitioner. Thus, for example, group sessions can be implemented or live supervision can be implemented in the experience room.

### BRIEF DESCRIPTION OF THE FIGURES

Further features and advantages of the invention will be apparent from the following, purely descriptive and in no way limiting, description of a preferred embodiment of the invention with reference to the accompanying drawings; therein showing:

FIG. 1 the schematic front view of an embodiment of the device for displaying experience spaces of a device for conducting serious games;

FIG. 2 schematic rear view of the device according to FIG. 1; and

FIG. 3 the schematic sequence of operation of the device for performing serious games.

### DETAILED DESCRIPTION OF THE INVENTION

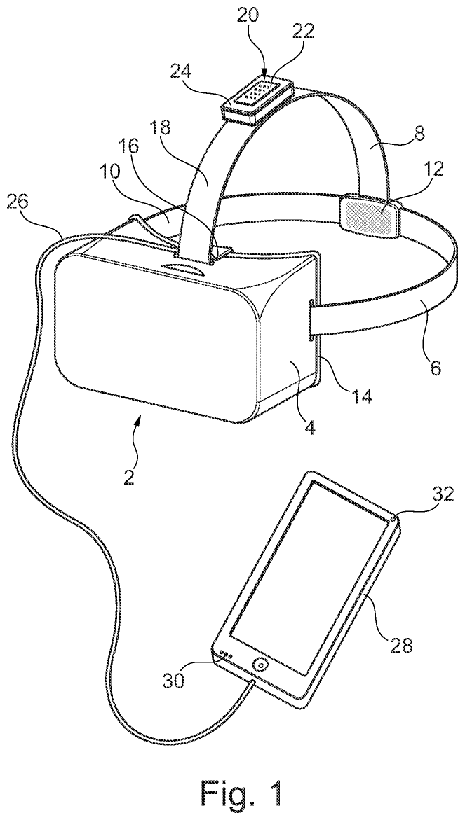

FIG. 1 shows a schematic front view of an embodiment of the device for displaying experience spaces of a device for performing serious games. As can be seen, the embodiment shown is a VR goggle 2, in this case comprising a screen or goggle portion 4 with a face pad 14 provided with three retaining straps 6, 8 and 10 for placement on a person's head to arrange the screen portion directly in front of the person's eyes. A three-way buckle 12 adjustably connects the three straps 6, 8 and 10 together.

On the face pad 14, in the illustrated embodiment, an optical heart rate sensor 16 is arranged for measuring the heart rate on the forehead of the person. From the heart rate sensor 16, a cable 18 extends on the upper tether 8 to a signal processing unit 20 comprising a signal processing chip 22 in a box 24 ultimately attached to the tether 8. Further, a USB cable 26 is provided for connection to a smartphone 28 which supplies power to the signal processing chip 22 and the heart rate sensor 16, with all data going from the into the signal processing chip 22 and then into the smartphone 28 via the USB interface 26.

The smartphone 28 is provided with (not shown) devices for the acquisition and storage of environmental data, in this case with microphones 30 and light sensors 32, with a module for the acquisition and storage of subjective raw data, a module for the acquisition and storage of therapeutic guide values, devices for the evaluation of the data of the heart rate sensor 16 and of the signal processing chip 22, of environmental data of the sensors 30 and 32 and the subjective raw data for calculating feedback coefficients, means for automated interpretation of the feedback coefficients taking into account the therapeutic guide values and deriving a feedback decision, and a module for controlling the VR goggles 2 in response to the feedback decision.

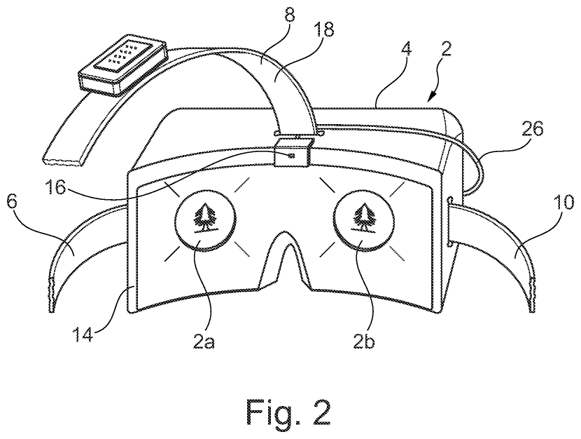

The FIG. 2 shows the rear view of the device according to FIG. 1, where same reference signs denote same elements. In particular, FIG. 2 shows the arrangement of the heart rate sensor 16 on the face pad 14, in such a way that the heart rate sensor 16 comes to rest on the forehead of the person wearing the VR goggles. In the embodiment shown, the heart rate sensor 16 is equipped with a light emitter and a receiver.

As can be further seen from FIG. 2, the VR goggles 2 are here provided with two screens 2a and 2b, for generating a stereo image for displaying experiential spaces, where these experiential spaces are generated by assigning certain media content to certain feedback coefficients, i.e., a certain image or movie to a certain stress level.

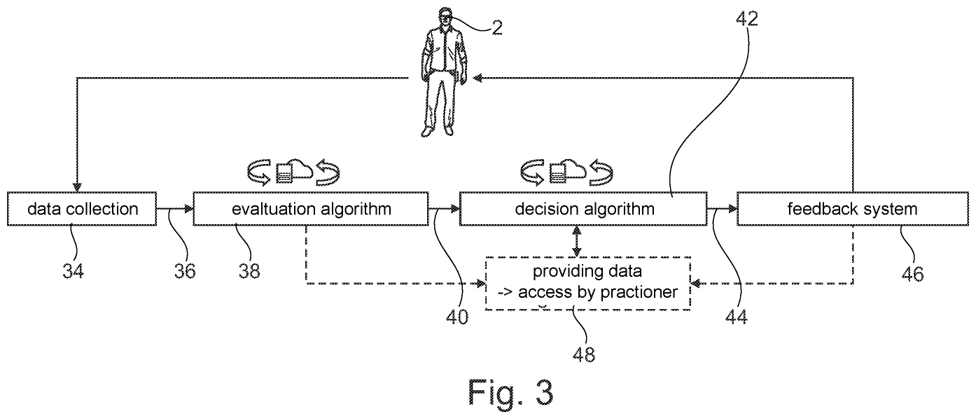

Finally, FIG. 3 shows in a schematic and illustrative manner the sequence of operation of the device according to FIGS. 1 and 2. for carrying out serious games according to the invention.

The first step 34 is data collection: entering subjective raw data in the self-report (questionnaires, condition report) and collecting body data with sensors in the VR goggles 2 (ECG—heart rate, HRV; EEG—e.g. PFC activity; EMG, SCL, eye tracking, head movements, gaze direction).

This raw data is passed on in a next step 36 for processing by an evaluation algorithm 38, which evaluates the data received in step 36 online at x second intervals and calculates a coefficient (e.g., tension coefficient, anxiety coefficient, pain coefficient . . . ).

The calculated coefficient or coefficients are then passed on in a step 40 for processing by a decision algorithm 42 to interpret the coefficients and derive a feedback decision (AI, learns from data from all sensors across interconnected devices according to the invention and all users).

The result of the interpretation of the coefficients by the decision algorithm 42 is passed as a derivative of a feedback decision in step 44 to a feedback system 46 for outputting sensory feedback via the VR goggles 2 based on decision of the decision algorithm 42 (auditory, visual, haptic, electrical, change of conditions of a software e.g. increase of difficulty, feedback to external software—e.g. e-mail to practitioner, triggering of a notification).

Data can also be permanently analyzed and shared with a practitioner in a medical setting, see reference numeral 48, the practitioner can influence the decision algorithm 42 if needed or initiate feedback manually via the feedback system 46.

What is claimed is:

1. A device for the implementation of serious games for the prevention and/or treatment of mental disorders, comprising
a. At least a facility for the representation of experiential spaces,
b. Sensors for physiological measurement of body functions,
c. Facilities for collecting and storing environmental data,
d. a module for recording and storing subjective raw data,
e. a module for recording and storing therapeutic guideline values,
f. Facilities for evaluating sensor and environmental data and raw subjective data to calculate feedback coefficients,
g. Facilities for automated interpretation of feedback coefficients, taking into account therapeutic benchmarks and deriving a feedback decision,
h. A module to control the facilities to display experiential spaces in response to the feedback decision,
i. Facilities for external monitoring of sensor data and feedback decisions, and
j. Facilities for external manipulation of feedback decisions.

2. The device according to claim 1, characterized in that the devices for calculating feedback coefficients are configured in such a way that the feedback coefficients are calculated repetitively at defined time intervals.

3. The device according to claim 1, characterized in that the sensors are those for measuring psychophysiological activities, selected from the group:
a. Cardiovascular activities, such as heart rate, heart rate variability, respiration/oxygenation of blood via electrocardiogram (ECG) or optical sensor;
b. Brain activities, such as electrical events of cortical regions of the brain, electroencephalography (EEG);
c. Muscle activity, such as muscle tension, respiratory activity, electromyography;
d. Electrodermal activity, such as sweat, skin conductance;
e. Eye activities, such as eye movements and gaze direction, eye tracking optically and with electrooculography (EOG);
f. Motion activities, such as head movements, upper body movements via motion sensors; and
g. Measurement of voice pattern and breath sounds.

4. The device according to claim 1, characterized in that the means for displaying experience spaces comprise elements for providing audiovisual content and/or sensory stimuli.

5. The device according to claim 1, characterized in that the experience space display means comprises at least one audiovisual content generator.

6. The device according to claim 1, characterized in that a media library for storing audiovisual as well as sensory media resources is provided for playback via the devices for displaying experience spaces, wherein certain media contents are assigned to certain feedback coefficients.

7. The device according to claim 1, characterized in that the devices for evaluating the sensor and environmental data and the subjective raw data for calculating feedback coefficients, and the device for automated interpretation of the feedback coefficients and derivation of a feedback decision are each configured as a central device to which a plurality of devices have access for use in the therapy of anxiety disorders, wherein feedback coefficients as well as feedback decision are calculated on the basis of aggregated data of all connected devices.

8. The device according to claim 1, characterized in that the means for evaluating the sensor and environmental data and the raw data and for calculating feedback coefficients, and the means for automatically interpreting the feedback coefficients and deriving a feedback decision are configured that an approximation to predefined target parameters is made by a systematic, iterative variation of the feedback decision in response to successive feedback coefficients.

9. The device according to claim 7, characterized in that the identification of the respective sensor data as well as the subjective raw data for the calculation of the feedback coefficients is performed by a self-learning pattern recognition system accessing the data of the central device.

10. The device of claim 9, characterized in that the pattern recognition is performed by AI methods, clustering analysis, machine learning, and/or the use of artificial neural networks.

11. The device according to claim 8, characterized in that the identification of the respective sensor data as well as the subjective raw data for the calculation of the feedback coefficients is performed by a self-learning pattern recognition system accessing the data of the central device.

12. The device of claim 11, characterized in that the pattern recognition is performed by AI methods, clustering analysis, machine learning, and/or the use of artificial neural networks

13. The device according to claim 1, characterized in that the devices for displaying experience spaces are designed as VR goggles, which are provided with sensors for physiological measurement of body functions.

14. The device according to claim 1, characterized in that the devices for displaying experience spaces are designed as simulators in which sensors for physiological measurement of body functions are provided.

15. The device according to claim 1, characterized in that the devices for displaying experience spaces are designed as augmented reality or mixed reality glasses, which are provided with sensors for physiological measurement of body functions.

16. The device according to claim 1, characterized in that the module for inputting subjective raw data is selected from the group consisting of recording voice commands and/or ambient sounds, recognizing directions of gaze, or recognizing gestures, querying multiple choice questions, querying binary indications, or indicating a range.

17. The device according to claim 1, characterized in that the means for analyzing the sensor and environmental data and the subjective raw data comprises means for analyzing speech and breathing sounds.

18. The device according to claim 1, characterized in that the environmental data acquisition devices comprise light sensors, cameras, motion sensors, accelerometers, and gyroscopes.

19. The device according to claim 1, characterized in that the device is locally or remotely connected to a plurality of devices of the same type via respective interfaces, for presenting an experience space common to all devices.
